(19) United States
(12) Patent Application Publication
(10) Pub. No.: US 2018/0064572 A1
(43) Pub. Date: Mar. 8, 2018

(54) UROSTOMY APPLIANCE

(71) Applicant: Salts Healthcare Limited, Aston, Birmingham West Midlands (GB)

(72) Inventor: Neil Wiltshire, Aston, Birmingham West Midlands (GB)

(21) Appl. No.: 15/554,405

(22) PCT Filed: Feb. 12, 2016
(86) PCT No.: PCT/GB2016/050348

§ 371 Date: Aug. 29, 2017

(30) Foreign Application Priority Data
Mar. 2, 2015 (GB) 1503472.1

(51) Int. Cl. A61F 5/44; A61F 5/445

(52) U.S. Cl. CPC A61F 5/4407; A61F 5/4405; A61F 5/445

(57) ABSTRACT

A urostomy appliance having:—first and second walls connected to each other at or near their peripheries, the first wall having a stoma-receiving opening; a urine collecting cavity defined between the first and second walls; and a connection member connected to the first wall for attaching the appliance to a user, wherein the appliance includes a stiffening member for preventing twisting of the appliance during use.

The invention relates to a urostomy appliance for collecting urine. It should be understood that the invention can be utilised in drainable and non-drainable urostomy appliances. The invention is also applicable to both one piece and two piece urostomy appliances.

According to a first aspect of the invention, we provide a urostomy appliance having:—

first and second walls connected to each other at or near their peripheries, the first wall having a stoma-receiving opening;

a urine collecting cavity defined between the first and second walls; and

a connection member connected to the first wall for attaching the appliance to a user,

wherein the appliance includes a stiffening member for preventing twisting of the appliance during use.

Further features of the first aspect of the invention are set out in claims 2 to 21 appended hereto.

Embodiments of the invention will now be described by way of example only with reference to the accompanying drawings, of which:—

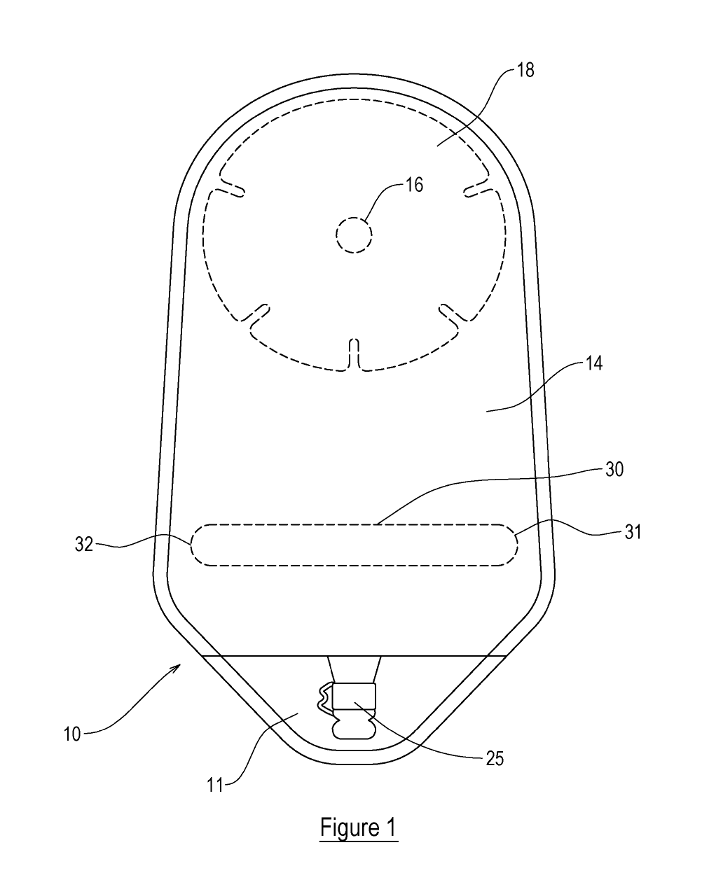

FIG. 1 is a front view of a first embodiment of a urostomy appliance in accordance with the present invention;

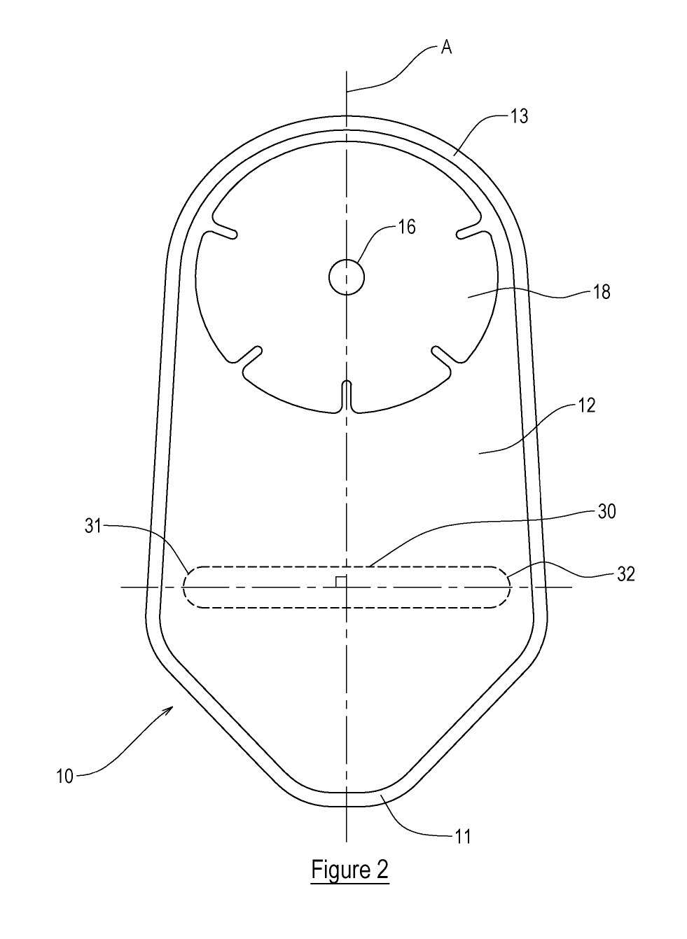

FIG. 2 is a rear view of the appliance of FIG. 1;

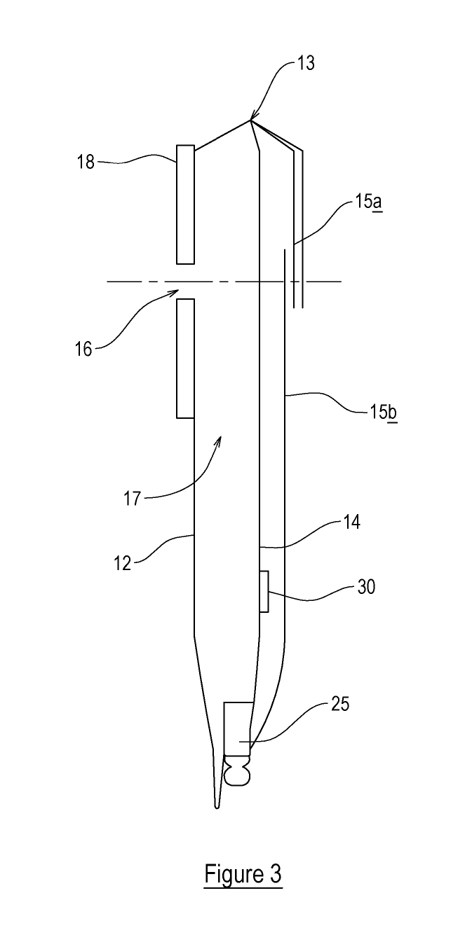

FIG. 3 is a cross-sectional view of the appliance of FIGS. 1 and 2 through the plane A; and

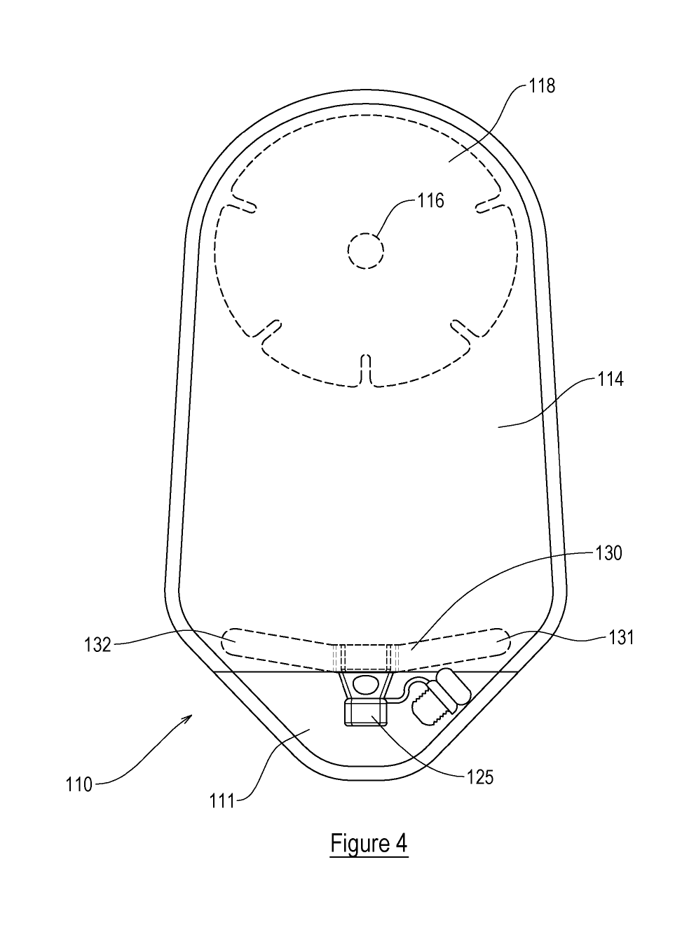

FIG. 4 is a front view of a second embodiment of a urostomy appliance in accordance with the present invention.

Referring firstly to the figures, these show a first embodiment of a urostomy appliance in accordance with the present invention, shown generally at 10. The general construction of the urostomy appliance 10 is similar to those well known in the art and in that sense it includes first 12 and second 14 walls connected to each other at or near their peripheries 13, for example by heat welding or using an adhesive. The walls 12, 14 are opaque other than where indicated below, although that need not necessarily be the case. The urostomy appliance shown is a drainable appliance, meaning that its contents can be emptied through a drainage outlet 25. In alternative embodiments, the appliance need not be drainable. The first 12 and second 14 walls define a urine collecting cavity 17.

The present embodiment also includes a further, third, wall made from upper 15a and lower 15b wall portions which overlap each other. The purpose of the third wall is to conceal the contents of the appliance during use, whilst permitting a user to view the contents through the overlapping wall portions 15a, 15b. The wall portions 15a, 15b are connected to the second wall at or near their peripheries 13.

The first wall 12 has a stoma-receiving opening 16 and is connected to a generally connecting member in the form of a circular flange 18 which is manufactured from a hydrocolloid material, for adhering the appliance 10 to a user around their stoma. In other embodiments, the connecting member may be part of a two-piece or the like coupling, with another connecting member being adhered to a user. Such alternatives of connecting the appliance to a user are well known, and so will not be discussed in further detail herein.

Advantageously, the appliance includes a stiffening member 30 which is connected to an exterior surface of the second wall 14. The stiffening member 30 is elongate and extends transversely (i.e. laterally) across the appliance and the second wall 14, substantially perpendicularly to the elongate axis A of the appliance 10. This position of the stiffening member 30 is not essential (it could extend at other angles) but is preferred to achieve maximum inhibition of twisting during use.

In alternative embodiments a stiffening member may be connected to the first wall in addition to or as an alternative to the second wall. In envisaged embodiments a stiffening member may be connected to the interior or exterior surface of the first and/or second wall. In envisaged embodiments, an appliance in accordance with the invention may have a receiving formation to receiving a stiffening member. For example one of the walls may include a pocket into which a stiffening member can be inserted. In a further example, the stiffening member and/or a wall of the appliance may be provided with an adhesive such that a user can connect the stiffening member to the wall.

The stiffening member 30 is positioned in a lower half of the appliance 10, such that it is positioned close to or adjacent a bottom of the appliance 10 (“bottom” being the end where urine collect under gravity). In this embodiment, which includes a drainage outlet 25, the stiffening member 30 is positioned close to or adjacent the drainage outlet 25. In this position the stiffening member 30 is particularly advantageous in inhibiting twisting of the drainage outlet 25 relative to a remainder of the appliance during use. Such twisting of the drainage outlet can occur at night, when the outlet is connected, via a suitable conduit, to a further collection container. Twist can give rise to increase discomfort, but more importantly, can cause leakages from the appliance.

The stiffening member 30 has opposite end portions 31, 32 which are semi-circular (i.e. part circular). The opposing end portions 31, 32 are positioned close to or adjacent the peripheries 13 of the first and second walls 12, 14. This ensures that the stiffening member 30 is as long as possible to provide maximum inhibition to twisting of the appliance during use. The part-circular shape of the end portions means that there are no sharp edges/points on the stiffening member 30 which could dig in to a user, particular when wearing the appliance whilst sleeping. In alternative embodiments the opposing end portions terminate at or adjacent the peripheries of the first and second walls. In yet further embodiments a stiffening member could extend around the entirety of the first and second walls.

Referring to FIG. 4, this shows a second embodiment of a urostomy appliance 110 in accordance with the present invention. Features in common with the first embodiment have been given the same reference numeral, but with the addition of 100. Those common features will not be discussed I further detail.

The appliance 110 also includes a stiffening member 130 which is connected to an exterior surface of the second wall 114. The stiffening member 130 is formed as an integral of the drainage outlet 125 with extension portions 131, 132 which are elongate and extend transversely (i.e. laterally) away from the outlet 125, substantially perpendicularly to the elongate axis of the appliance 110 towards peripheries of the wall 114. This position of the stiffening member 130 is not essential (it could extend at other angles) but is preferred to achieve maximum inhibition of twisting during use.

In alternative embodiments the stiffening member 130 may be connected to the first wall in addition to or as an alternative to the second wall 114. In envisaged embodiments the stiffening member 130 may be connected to the interior or exterior surface of the first and/or second wall 114.

It should be appreciated that whilst the stiffening member 130 is integral with the outlet 125, it may be a separate component part, but positioned in the same or a similar location at or near the outlet 125. Likewise, the apparatus 110 may be provided with only one of the extension portions 131, 132, or additional extension portions to those (e.g. three or more).

Whilst in the present embodiments the stiffening members 30, 130 are substantially planar, embodiments are envisaged where the stiffening member may be convex such that it urges the wall to which it is connected away from the other wall/cavity. In such embodiments the convex shape assists in further inhibition of twisting of the appliance, and particular the drainage outlet, during use.

When used in this specification and claims, the terms “comprises” and “comprising” and variations thereof mean that the specified features, steps or integers are included. The terms are not to be interpreted to exclude the presence of other features, steps or components.

The features disclosed in the foregoing description, or the following claims, or the accompanying drawings, expressed in their specific forms or in terms of a means for performing the disclosed function, or a method or process for attaining the disclosed result, as appropriate, may, separately, or in any combination of such features, be utilised for realising the invention in diverse forms thereof.

1. A urostomy appliance having:—
first and second walls connected to each other at or near their peripheries, the first wall having a stoma-receiving opening;
a urine collecting cavity defined between the first and second walls; and
a connection member connected to the first wall for attaching the appliance to a user,
wherein the appliance includes a stiffening member for inhibiting twisting of the appliance during use.

2. A urostomy appliance according to claim 1 wherein the stiffening member is connected to one of the walls.

3. A urostomy appliance according to claim 1 wherein the stiffening member is held in a receiving formation provided on or by one of the walls.

4. A urostomy appliance according to claim 1 wherein the stiffening member is connected to the first wall.

5. A urostomy appliance according to claim 4 wherein the stiffening member is connected to an exterior surface of the first wall.

6. A urostomy appliance according to claim 4 wherein the stiffening member is connected to an interior surface of the first wall.

7. A urostomy appliance according to claim 1 wherein the stiffening member is connected to the second wall.

8. A urostomy appliance according to claim 7 wherein the stiffening member is connected to an exterior surface of the second wall

9. A urostomy appliance according to claim 7 wherein the stiffening member is connected to an interior surface of the second wall

10. A urostomy appliance according to claim 1 wherein the stiffening member extends transversely across the wall.

11. A urostomy appliance according to claim 1 wherein the stiffening member extends laterally across the wall.

12. A urostomy appliance according to claim 1 wherein the stiffening member extends substantially perpendicularly to a longitudinal axis of the appliance.

13. A urostomy appliance according to claim 1 wherein the stiffening member is positioned in a lower half of the appliance.

14. A urostomy appliance according to claim 1 wherein the stiffening member is positioned close to or adjacent a bottom of the appliance.

15. A urostomy appliance according to claim 1 wherein the appliance includes a drainage outlet, and wherein the stiffening member inhibits twisting of the drainage outlet relative to a remainder of the appliance during use.

16. A urostomy appliance according to claim 15 wherein the stiffening member is positioned close to or adjacent the drainage outlet.

17. A urostomy appliance according to claim 1 wherein the stiffening member is elongate.

18. A urostomy appliance according to claim 1 wherein the stiffening member includes opposing end portions which are positioned close to or adjacent the peripheries of the first and second walls.

19. A urostomy appliance according to claim 1 wherein the stiffening member includes opposing end portions which terminate at or adjacent the peripheries of the first and second walls.

20. A urostomy appliance according to claim 1 wherein the end portions of the stiffening member are curved.

21. A urostomy appliance according to claim 1 wherein the end portions of the stiffening member are part-circular.

22. A urostomy appliance according to claim 1 wherein the stiffening member is convex such that it urges a first or second wall to which it is connected away from the other wall or the cavity

23. A urostomy appliance according to claim 15 wherein the stiffening member is connected to the drainage outlet or formed as an integral part of the drainage outlet.

24. A urostomy appliance according to claim 1 wherein the stiffening member is substantially planar.

25-26. (canceled)
